(19) United States
(12) Patent Application Publication
(10) Pub. No.: US 2006/0205061 A1
(43) Pub. Date: Sep. 14, 2006

(54) Biosensors based upon actuated desorption

(75) Inventor: Michael L. Roukes, Pasadena, CA (US)

Correspondence Address: BUCHANAN, INGERSOLL & ROONEY LLP, P.O. BOX 1404, ALEXANDRIA, VA, 22313-1404, US

(73) Assignee: California Institute of Technology, Pasadena, CA (US)

(21) Appl. No.: 11/287,109

(22) Filed: Nov. 23, 2005

Related U.S. Application Data
(60) Provisional application No. 60/630,921, filed on Nov. 24, 2004.

(51) Int. Cl. C12M 1/34

(52) U.S. Cl. 435/287.2

(57) ABSTRACT

Provided are methods and systems for a rational concatenation of specific classes of proven microfluidic-based protocols to enable a systems approach to repetitive, synchronous analyte detection via a pristine detection array under programmed and/or automatic control.

### CROSS REFERENCE TO RELATED APPLICATIONS

The application claims priority under 35 U.S.C. §119 to U.S. Provisional Application Serial No. 60/630,921, filed Nov. 24, 2004, the disclosure of which is incorporated herein by reference.

### TECHNICAL FIELD

The invention relates generally to methods and apparatus for conducting analyses, particularly fluidic devices for the detection of target analytes.

### BACKGROUND

There are a number of devices and assays for the detection of the presence and/or concentration of specific analytes in fluids and gases. Many of these rely on interactions between a binding agent and its cognate as the mechanism of detection. Interaction of the two members is typically measured by the detection of a detectable label associated with a particular member of the pair.

There is a significant trend to reduce the size of these sensors, for sensitivity,. to reduce reagent costs as well as to reduce the amount of sample needed. Thus, a number of microfluidic devices have been developed, generally comprising a solid support with microchannels, utilizing a number of different wells, pumps, reaction chambers, and the like. See for example EP 0637996 B1; EP 0637998 B1; WO96/39260; WO97/16835; WO98/13683; WO97/16561; WO97/43629; WO96/39252; WO96/15576; WO96/15450; WO97/37755; and WO97/27324; and U.S. Pat. Nos. 5,304,487; 5,071531; 5,061,336; 5,747,169; 5,296,375; 5,110,745; 5,587,128; 5,498,392; 5,643,738; 5,750,015; 5,726,026; 5,35,358; 5,126,022; 5,770,029; 5,631,337; 5,569,364; 5,135,627; 5,632,876; 5,593,838; 5,585,069; 5,637,469; 5,486,335; 5,755,942; 5,681,484; and 5,603,351.

### SUMMARY

The invention provides a rational concatenation of specific classes of proven microfluidic-based protocols to enable a systems approach to repetitive, synchronous analyte detection via a pristine detection array—under programmed and/or automatic control. The invention comprises the use of (a) microfluidics-based, sample preparation (which may include programmed steps of filtration and valved or electrochemical release of reagents), (b) analyte immobilization onto elements of an array designed to enable subsequent programmed (e.g., timed) release (e.g., specific and/or selective), and (c) repeated cycles of synchronous detection within a sensor chamber—located separate from preprocessing to preclude desensitization the prepatory process, and to allow for synchronized release-delay-gated detection cycles to reduce false positives. The elements of the array allow for averaging the gated response from individual cycles to enhance the signal-to-noise ratio of the detection process. This systems-based approach provides, for the first time, a route to high confidence detection by concatenating the benefits of affinity-based capture, pristine detection methods, with temporal knowledge of when “the detection signal” should occur, if present.

The invention provides a system for the detection of a target analyte in a fluid sample. The system comprising a capture chamber having an inlet port; an outlet port; and at least one solid support member, wherein the solid support member is disposed between and in fluid communication with the inlet and outlet port, the at least one solid support member comprising at least one binding ligand specific for the target analyte; a sample reservoir, fluidly connected to the inlet port; a labeled binding ligand reservoir, fluidly connected to the inlet port; a detector, fluidly connected to the outlet port; and means for controlling the timed release and flow of a binding ligand from the at least one solid support to the detector.

The invention also provides a method for the detection of a target analyte in a fluid sample. The method includes contacting the sample with at least one solid support member comprising at least one first binding ligand removably attached to the solid support, wherein the at least one first binding ligand is specific for the target analyte, wherein the target analyte interacts with the binding ligand to form a bound analyte; contacting the bound analyte with a second binding ligand comprising a label, wherein the second binding ligand interacts with the target analyte to form a complex; dissociating the complex from the solid support; and detecting the label on the complex.

The details of one or more embodiments are set forth in the accompanying drawings and the description below. Other features, objects, and advantages will be apparent from the description and drawings, and from the claims.

### BRIEF DESCRIPTION OF THE FIGURES

FIG. 1 shows an illustrative method of the invention.

FIG. 2A-D is a schematic depicting a solid surface comprising a binding ligand (A) upon exposure to analyte, (B) upon exposure to a labeled binding ligand (C) following washing of non-bound labeled binding ligand, and (D) following release of a complexed analyte from the solid surface.

FIG. 3A and B are schematics showing a system of the invention. (A) shows the general overall structure of the device of the invention. (B) depicts the timed release and subsequent detection of the target analytes. A pulsatory stimulus—for example, electrical or optical—is applied to a given capture pad. The released label/analyte/immobilization-entity complex flows to the detector in a precalibrated time, and is observed using a detection process synchronized to the release stimulus, delayed by a pre-calibrated amount. This delay may simply be based upon the average flow velocity of particles in solution and the pre-measured array-detector path length. Alternatively, this may be directly measured by a test release from a calibration pad incorporated within each capture array. The synchronous detection process is illustrated in the top left inset.

The figures are exemplary only; other embodiments are within the following description and claims. The invention, together with further objects and advantages, must be understood by reference to the following description taken in conjunction with the accompanying drawings in the several figures of which like reference numerals identify like elements

### DETAILED DESCRIPTION

As used herein and in the appended claims, the singular forms “a,” “and,” and “the” include plural referents unless the context clearly dictates otherwise. Thus, for example, reference to “an analyte” includes a plurality of such analytes and reference to “the valve” includes reference to one or more valves known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

The invention provides methods and system for analyte detection, quantification and identification. The invention provides methods of eliminating contaminants prior to detection as well as optimizing the sensitivity of the system to facilitate analyte detection, quantification and identification.

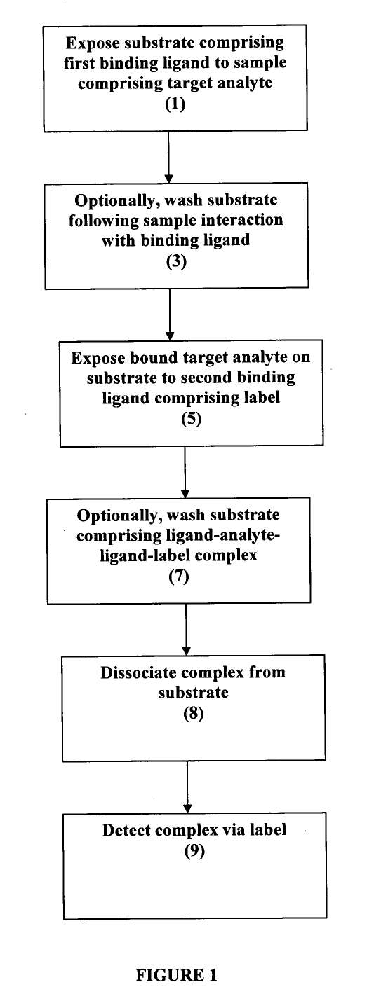

A representative method of the invention is depicted in FIG. 1. The methods and systems of the invention comprise the process of contacting 1 a first binding ligand removably immobilized on a solid surface with a sample comprising a target analyte. If the target analyte is present in the sample, the binding ligand will capture the target analyte. Unbound sample analytes can be removed from the bound sample analyte using, for example, an optional wash step 3. The analyte is also exposed 5 to a second binding ligand comprising a detectable label. The analyte in the sample may be exposed to the labeled binding ligand either before or after exposure to the binding ligand immobilized on the solid surface such that a complex labeled analyte is obtained. The bound and labeled analyte can then be washed 7 clear of non-bound label or other contaminants. The complexed-labeled analyte is then dissociated 8 from the solid surface using any number of techniques (described more fully below). The dissociated bound-labeled analyte is then detected 9 using a detection method outlined below, including, for example, resistometric, mechanical, optical, or magnetic detection.

Biosensors based upon electrically-actuated immunospecific desorption (EAID) process as identified herein are further described. The process comprises, in one aspect, capture of the target analyte from solution (e.g., immunospecific capture). Capture and immobilization of the target analyte can be accomplished, for example, by antibodies or aptamers or, in the case of nucleic acid analytes, by complementary oligomers. The capture entities (e.g., binding ligands) themselves can be immobilized by various chemistries and physical properties such as direct derivatization with biotin, and linkage with streptavidin functionalized alkanethiols bound to gold pads or with dielectrophoretic forces. Dielectrophoretic forces can also be used to enhance the rate of capture (e.g., to speed up the rate at which the immobilized capture agents acquire the cognate analytes.)

The process continues by exposing the captured ligand to a second binding ligand comprising a label. The second capture process can also employ agents such as used in the first process, and these could bind to a second epitope on the target analyte, or on similar epitope if the analyte is multivalent. The label is formed by an entity that, for example, increases the average conductivity of the solution or perhaps by providing a high level of local bound charge to the medium. Alternatively it could be an entity with its own surface bound molecules that are desorbed and detected during the detection process.

Once a complex (e.g., a ligand-analyte-ligand-label complex) is formed, timed (e.g., a pulsatory) electrochemical or photochemical release of the immobilized label/analyte/ligand complex is performed. This can be performed by, for example, a simple electrocleaning step by raising the electrochemical potential of the capture solid surface to the point where the immobilization entity becomes detached from the solid surface or, alternatively, by photochemical dissociation of a specific linker molecule specifically incorporated into the immobilization entity for this purpose. Where the binding ligand is linked to the solid surface by a cleavable peptide or nucleic acid, enzyme desorption (e.g., restriction enzymes and the like) may be utilized.

Detection of the complex is then obtained through any number of methods (e.g., a gated electrical detection of the presence of the labeled analyte). This can be achieved by electrical means, either through conductance, capacitance or charge based detection. Alternatively it could be achieved optically, by a local optical stimulus and subsequent detection, e.g. through fluorescence. Knowledge of the temporal “window” for arrival of the species provides significant rejection of false positives. The temporal location of this “window” is based upon knowledge of the time the analyte complex is released, of the transit time required for fluidic transport along the channels leading from the capture pad to the detector assembly, and of the typical variance of this transit time.

Gated, or “boxcar”, detection is a historical method that allows rejection of detector responses that are temporally unrelated to a stimulus provided. It presupposes one has knowledge of the time delay involved in onset the response, and the temporal duration of the response. Based on such information one rejects signals that are acquired outside a temporal “window” in which coherent response is deemed unlikely. In the present invention local electrochemical, optical, or other methods are used to release analytes at a specific time from a specific capture pad/support within the capture chamber. Programmed flow rates within the microfluidics channel connecting the capture and detection chamber—given the conditions of laminar flow—yield a highly reproducible transit time for the released complex to arrive at the sensors within the detection chamber. Furthermore, foreknowledge of the temporal signature of detection—established by initial calibration procedures—allows setting the duration of the detection process. Thus a time window is formed, bounded by the arrival and detection times, outside of which all signals acquired are deemed unrelated to the process of specific analyte detection. This ability to reject spurious signals, in other words to limit the signal averaging process to intervals over which the probability of detection is finite, greatly enhances the signal-to-noise level of detection.

A microfluidic flow regulator can be used in the system and methods of the invention. The apparatus also includes a microfluidic flow regulator, such as one or more of micropumps described herein, for controlling the flow rate, for example, the pump may be a microelectromechanical (MEMS) microfluidic pump. The micropump can be operated at a predetermined frequency, which can be either substantially constant or modulated depending upon the requirements of the system, the temporal release issues and detector measurement timing and the like.

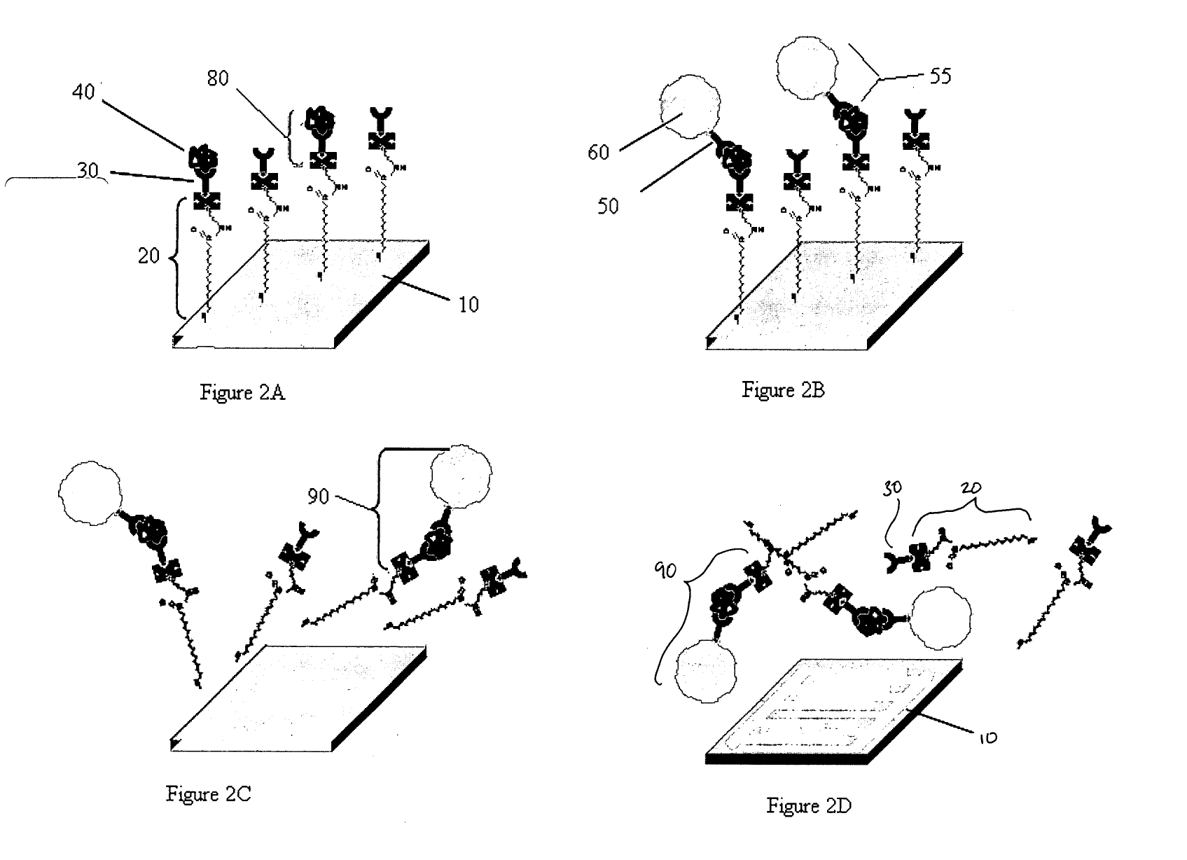

FIG. 2A-D shows a solid surface comprising a binding ligand in more detail at various points in a method of the invention. Solid surface 10 can be any number of materials useful to immobilize an agent (e.g., the agent can be a polypeptide, nucleic acid, chemical moiety, and the like). Also depicted is a linker 20, which attaches binding ligand 30 to solid surface 10. The linker 20 may be any type of interaction between the solid surface 10 and binding ligand 30 including, but not limited to, a covalent bond, a chemical bond, an electrostatic interaction, a photochemical interaction and the like, as more fully described below.

In FIG. 2A the binding ligand, removably bound to the solid surface, is exposed to a sample (e.g., target) analyte 40. Analyte 40 is capable of binding to or interacting with binding ligand 30. In one aspect of the invention, once analyte 40 has been exposed to the binding ligand 30 for a sufficient amount of time and under conditions which allow for the interaction of the analyte 40 with the binding ligand 30, the solid surface comprising bound analyte 80 (see, e.g., FIG. 2A) is washed to remove any contaminants and non-bound analyte.

In FIG. 2B, bound analyte 80 is exposed to a labeled binding domain 55 comprising a binding domain 50 and a detectable label 60. Binding domain 50 is capable of binding to or interacting with analyte 40. In one aspect of the invention, once labeled binding domain 55 has been exposed to the bound analyte 80 for a sufficient amount of time and under conditions which allow for the interaction of the analyte 40 with the labeled binding domain 55, the solid surface comprising analyte complex 90 is washed to remove any contaminants and non-bound labeled binding domains 55. Once the analyte complex 90 is obtained, the analyte complex can be dissociated from solid surface 10 (see, e.g., FIG. 2D) using electrochemical techniques, photochemical techniques and the like (as described more fully below).

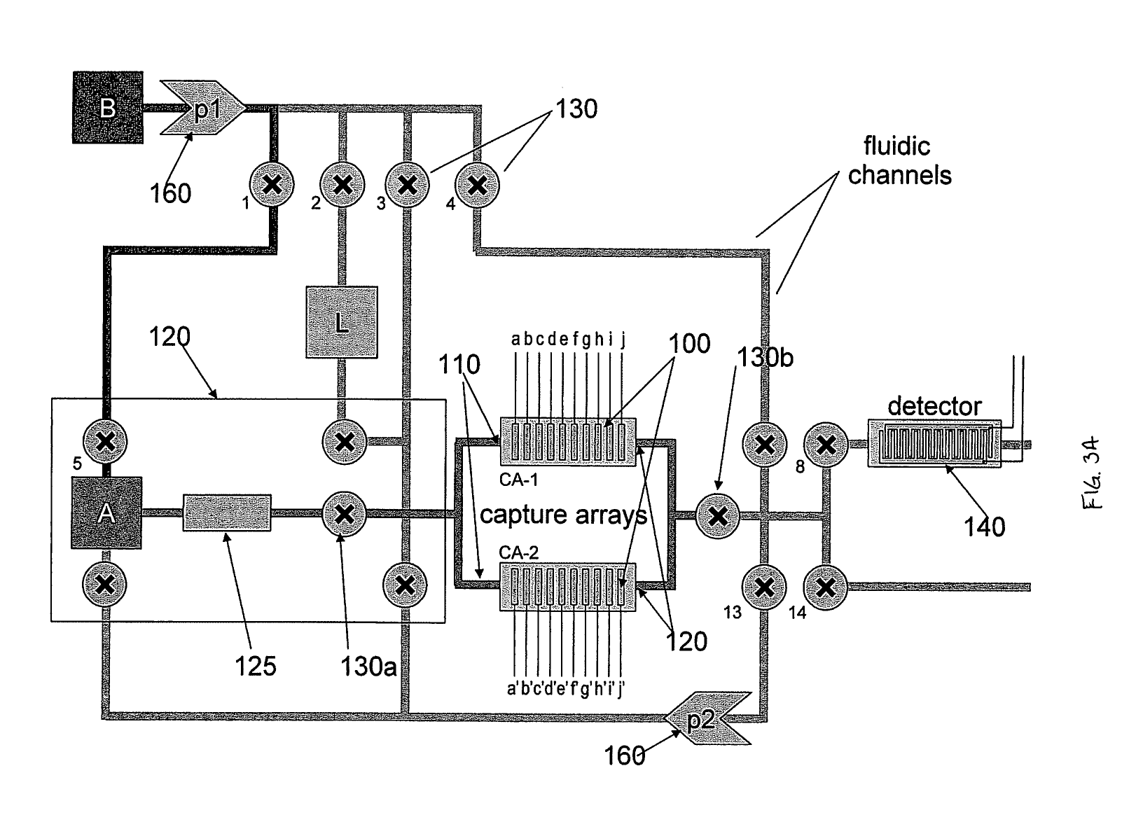

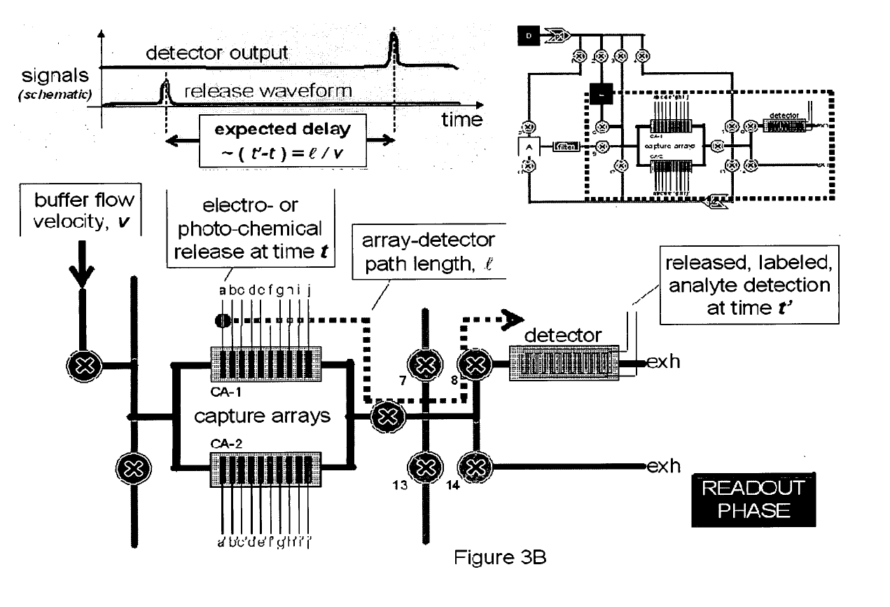

In one aspect of the invention, the release of a complex from the solid support is performed such that the timing of detection is dependent upon the flow period from the location of the bound ligand to the detector. In this manner, the detector is selectively actuated at the period in which a labeled complex will be present in the detection chamber. Using this technique noise and contaminant measurements are reduced. The timing of the control of the release and detector can be controlled manually or by a computer. The computer, for example, can control the release of an enzyme from a storage reservoir, wherein the enzyme cleaves the bound ligand-complex from the solid support. In another aspect, the computer can control the release from the solid support by modulating the electrical properties of the solid support. Furthermore, the computer can control the fluid flow rate by modulating, for example, a pump's flow in the system. In addition, the computer can control the timing of detection (e.g., photoactuation for fluorescence detection and the like; depending upon the detectable label used in the methods). The timing of detection can be calculated based upon at least 2 basic parameters (i) the flow rate in the system, and (ii) the distance from the location of the binding ligand-complex to the detector. Using these parameters, the temporal detection of the complex can be determined and optimized as depicted in FIG. 3B.

When the target analyte 40 is bound by the binding ligand 30, the complex 90 may be released for detection purposes, if necessary, using any number of known techniques, depending on the type of the interaction a binding ligand or linking moiety has with the solid surface, including changes in pH, salt concentration, temperature, enzymatic action, electronic charge and the like.

A solid surface 10 suitable for the attachment of binding ligands (e.g., biological molecules) and the performance of molecular interaction assays may be made of any suitable material including, but not limited to, metal, glass, and plastic and may be modified with coatings (e.g., metals or polymers). The solid support can be a metal, glass or silicon surface. In a one embodiment, the solid substrate can be made from a wide variety of materials, including, but not limited to, silicon such as silicon wafers, silicon dioxide, silicon nitride, glass and fused silica, gallium arsenide, indium phosphide, aluminum, ceramics, polyimide, quartz, plastics, resins and polymers including polymethylmethacrylate, acrylics, polyethylene, polyethylene terepthalate, polycarbonate, polystyrene and other styrene copolymers, polypropylene, polytetrafluoroethylene, superalloys, zircaloy, steel, gold, silver, copper, tungsten, molybdeumn, tantalum, KOVAR, KEVLAR, KAPTON, MYLAR, brass, sapphire, and the like. High quality glasses such as high melting borosilicate or fused silicas may be used for their UV transmission properties when any of the sample manipulation steps require light based technologies. In addition, portions of the device may be coated with a variety of coatings as needed, to reduce non-specific binding, to allow the attachment of binding ligands, for biocompatibility, for flow resistance, and the like. In some embodiments of the invention, binding ligands are immobilized to solid surfaces such as, for example, beads, microspheres, the bottoms of microwells, microchannels and the like.

In some aspects of the invention the solid surface may be modified to attach binding ligands (e.g., biological molecules) in an array format (e.g., a microarray). As used herein, the term “microarray” refers to a solid surface comprising a plurality of addressed or addressable binding ligands (e.g., nucleic acids, peptides, polypeptide and the like). The location of each of the binding ligands or groups of binding ligands in the array is typically known, so as to allow for identification and, as more fully described below, selective release of the binding ligand and/or an analyte complex to assist in the temporal identification of the release complex at the detector.

Examples of binding ligands (e.g., biological molecules) that can be used in the methods and systems of the invention include molecules (e.g., polymers) typically found in living organisms. Examples include, but are not limited to, proteins, nucleic acids, lipids, and carbohydrates.

As used herein, the term “target analyte” refers to a molecule in a sample to be detected. Examples of target analytes include, but are not limited to, polynucleotides, oligonucleotides, viruses, polypeptides, antibodies, naturally occurring drugs, synthetic drugs, pollutants, allergens, affector molecules, growth factors, chemokines, cytokines, and lymphokines.

Analytes include organic and inorganic molecules, including biological molecules. For example, the analyte may be an environmental pollutant (including pesticides, insecticides, toxins, and the like); a chemical (including solvents, polymers, organic materials, and the like); therapeutic molecules (including therapeutic and abused drugs, antibiotics, and the like); biological molecules (including, e.g., hormones, cytokines, proteins, lipids, carbohydrates, cellular membrane antigens and receptors (neural, hormonal, nutrient, and cell surface receptors) or their ligands); whole cells (including prokaryotic and eukaryotic cells; viruses (including, e.g., retroviruses, herpesviruses, adenoviruses, lentiviruses); spores; and the like.

Suitable proteinaceous target analytes include, but are not limited to, immunoglobulins, particularly IgEs, IgGs and IgMs, and particularly therapeutically or diagnostically relevant antibodies, including but not limited to, antibodies to human albumin, apolipoproteins (including apolipoprotein E), human chorionic gonadotropin, cortisol, α-fetoprotein, thyroxin, thyroid stimulating hormone (TSH), antithrombin, antibodies to pharmaceuticals (including antieptileptic drugs (phenytoin, primidone, carbariezepin, ethosuximide, valproic acid, and phenobarbitol), cardioactive drugs (digoxin, lidocaine, procainamide, and disopyramide), bronchodilators (theophylline), antibiotics (chloramphenicol, sulfonamides), antidepressants, immunosuppresants, abused drugs (amphetamine, methamphetamine, cannabinoids, cocaine and opiates) and antibodies to any number of viruses (including orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavinus), polyomaviruses, and picornaviruses, and the like), and bacteria (including a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprea; Clostridium, e.g. C. botulinum, C. teteni, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lamblia, Y. pestis, Pseudomonas, e.g. P. aeruginosa, P. putida; Chlamydia, e.g. C. trachomatis; Bordetella, e.g. B. pertussis; Treponema, e.g. T. palladium; and the like); enzymes (and other proteins), including, but not limited to, enzymes used as indicators of or treatment for heart disease, including creatine kinase, lactate dehydrogenase, aspartate amino transferase, troponin T, myoglobin, fibrinogen, cholesterol, triglycerides, thrombin, tissue plasminogen activator (tPA); pancreatic disease indicators including amylase, lipase, chymotrypsin and trypsin; liver function enzymes and proteins including cholinesterase, bilirubin, and alkaline phosphotase; aldolase, prostatic acid phosphatase, terminal deoxynucleotidyl transferase, and bacterial and viral enzymes such as HIV protease; hormones and cytokines (many of which serve as ligands for cellular receptors) such as erythropoietin (EPO), thrombopoietin (TPO), the interleukins (including IL-1 through IL-17), insulin, insulin-like growth factors (including IGF-1 and -2), epidermal growth factor (EGF), transforming growth factors (including TGF-α and TGF-β), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cotrisol, estradiol, follicle stimulating hormone (FSH), thyroid-stimulating hormone (TSH), leutinzing hormone (LH), progeterone and testosterone; and (4) other proteins (including α-fetoprotein, carcinoembryonic antigen CEA, cancer markers, and the like). In addition, any of the biomolecules that are indirectly detected through the use of antibodies may be detected directly as well; that is, detection of virus or bacterial cells, therapeutic and abused drugs, and the like, may be done directly.

Suitable target analytes include carbohydrates, including, but not limited to, markers for breast cancer (CA15-3, CA 549, CA 27.29), mucin-like carcinoma associated antigen (MCA), ovarian cancer (CA125), pancreatic cancer (DE-PAN-2), prostate cancer (PSA), CEA, and colorectal and pancreatic cancer (CA 19, CA 50, CA242). Suitable target analytes also include metal ions, particularly heavy and/or toxic metals, including but not limited to, aluminum, arsenic, cadmium, selenium, cobalt, copper, chromium, lead, silver and nickel.

These target analytes may be present in any number of different sample types, including, but not limited to, bodily fluids including blood, lymph, saliva, vaginal and anal secretions, urine, feces, perspiration and tears, and solid tissues, including liver, spleen, bone marrow, lung, muscle, brain, and the like. For example, in its broadest sense a sample includes, but is not limited to, environmental, industrial, and biological samples. Environmental samples include material from the environment such as soil and water. Industrial samples include products or waste generated during a manufacturing process. Biological samples may be animal, including, human, fluid (e.g., blood, plasma and serum), solid (e.g., stool), tissue, liquid foods (e.g., milk), and solid foods (e.g., vegetables).

Binding ligands, partners or cognates refers to two molecules (e.g., proteins) that are capable of, or suspected of being capable of, physically interacting with each other. As used herein, the terms “first binding ligand” and “second binding ligand” refer to two binding ligands that are capable of, or suspected of being capable of, physically interacting with each other. Two nucleic acid molecules capable of hybridizing to one another due to complementarity are to be understood as binding partners where the context is appropriate.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

In one aspect of the invention, a solid surface comprising a binding ligand is located within a fluidic device that can be used to affect a number of manipulations of a sample to result in target analyte detection or quantification. Such manipulations can include cell lysis, cell removal, cell separation, and the like, separation of the desired target analyte from other sample components, chemical or enzymatic reactions on the target analyte, detection of the target analyte and the like. The devices of the invention can include one or more reservoirs for sample manipulation and storage, waste or reagent storage; fluid channels to and between such reservoirs, including microfluidic channels. Such channels may comprise electrophoretic separation systems (e.g., microelectrodes); valves to control fluid movement; pumps such as electroosmotic, electrohydrodynamic, or electrokinetic pumps; and detectors as more fully described herein. The devices of the invention can be designed to manipulate one or a plurality of samples or analytes simultaneously or sequentially.

In addition to the solid substrate 10 described above, with reference to FIG. 2, the devices of the invention are configured to include one or more of a variety of components, that will be present on any given device depending on its use. As shown in FIGS. 3, these components include, but are not limited to: sample inlet ports 110; sample introduction or collection modules 120; cell handling modules (for example, for cell lysis, cell removal, cell concentration, cell separation or capture, cell growth, and the like); modification chamber 125 for chemical or biological alteration of the sample, including amplification of the target analyte (for example, when the target analyte is nucleic acid, amplification techniques are useful, including, but not limited to polymerase chain reaction (PCR), ligase chain reaction (LCR), strand displacement amplification (SDA), and nucleic acid sequence based amplification (NASBA)), chemical, physical or enzymatic cleavage or alteration of the target analyte, or chemical modification of the target; fluid pumps 160; fluid valves 130; thermal modules for heating and cooling; storage modules for assay reagents; interaction chamber(s) 100; and detection modules 140.

Referring to FIG. 3 there is shown a system of the invention. From left to right the components are as follows: B=reservoir for buffer solution, A=reservoir comprising an analyte to be detected, L=reservoir for label/capture complex, circles encompassing (x)=electrically-actuated fluidic valves, heavy lines=fluidic channels, light lines=electrical connections, P1 and P2=electrically-actuated fluidic pumps, exh=fluidic exhaust ports. In this aspect of the invention a first fluid comprising a target analyte 40 is delivered through one port while a second fluid comprising an agent that interacts with the target analyte enters through the second port. Once both fluids are delivered to the interaction chamber 100 the target analyte 40 and agent interact.

The at least one inlet port 110 and one outlet port 120 can comprise valves 130a and 130b to control delivery and removal of a fluid from the interaction chamber 100. The interaction chamber can comprise solid substrate 10 comprising binding ligand 30. Following interaction of the target analyte 40 with binding ligand 30, the system can be washed, if desired, by opening at least one fluid port valve 130a and effluent valve 130b. In this way, the solid substrate 10 can be washed. Alternatively, or following washing, an analyte complex 90 formed by interaction of the analyte 40 with the binding ligand 30 can be dissociated and washed from the solid substrate 10. The analyte complex 90 can then be collected in detection module 140.

Thus, the devices of the invention include at least one flow channel that allows the flow of sample from an inlet port 110 or reservoir to the other components or modules of the system. As will be appreciated by those in the art, the flow channels may be configured in a wide variety of ways, depending on the use of the channel. For example, a single flow channel starting at the sample inlet port may be separated into a variety of smaller channels, such that the original sample is divided into discrete subsamples for parallel processing or analysis. Alternatively, several flow channels from different modules, for example, the sample inlet port and a reagent storage module may feed together into an interaction chamber 100. As will be appreciated by those in the art, there are a large number of possible configurations; what is important is that the flow channels allow the movement of sample and reagents from one part of the device to another. For example, the path lengths of the flow channels may be altered as needed; for example, when mixing and timed reactions are required, longer flow channels can be used.

In one embodiment, the devices of the invention include at least one inlet port 110 for the introduction of the sample to the device. This may be part of or separate from a sample introduction or sample mixing chamber; that is, the sample may be directly fed in from the sample inlet port to a chamber comprising the solid substrate, or it may be pretreated in a sample mixing chamber.

In another aspect of the invention, the devices of the invention may include a cell manipulation chamber. This is used when the sample comprises cells that either contain the target analyte or that must be removed in order to detect the target analyte. For example, the detection of a target analyte in blood can require the removal of the blood cells for efficient analysis, or the cells (and/or nucleus) must be lysed prior to detection. In this context, “cells” include eukaryotic and prokaryotic cells, and viral particles that may require treatment prior to analysis, such as the release of nucleic acid from a viral particle prior to detection of target nucleic acids.

The sample is then provided to the interaction chamber 100 comprising binding ligands 30, as is generally outlined herein for target analyte detection. In this embodiment, binding ligands are immobilized (either by physical absorption or covalent attachment, described elsewhere herein) within the interaction chamber 100. As will be appreciated by those in the art, the composition of the binding ligand will depend on the sample component to be separated. Binding ligands for a wide variety of analytes are known or can be readily found using known techniques. For example, when the component is a protein, the binding ligands include proteins (particularly including antibodies or fragments thereof (FAbs, and the like)) or small molecules. When the sample component is a metal ion, the binding ligand generally comprises traditional metal ion ligands or chelators. Typical binding ligand proteins include peptides. For example, when the component is an enzyme, suitable binding ligands include substrates and inhibitors. Antigen-antibody pairs, receptor-ligands, and carbohydrates and their binding partners are also suitable component-binding ligand pairs. The binding ligand may be nucleic acid, when nucleic acid binding proteins are the targets. As will be appreciated by those in the art, the composition of the binding ligand will depend on the composition of the target analyte. Binding ligands to a wide variety of analytes are known or can be readily found using known techniques.

For example, when the analyte is a single-stranded nucleic acid, the binding ligand is generally a substantially complementary nucleic acid. Similarly the analyte may be a nucleic acid binding protein and the capture binding ligand is either a single-stranded or double-stranded nucleic acid; alternatively, the binding ligand may be a nucleic acid binding protein when the analyte is a single or double-stranded nucleic acid. When the analyte is a protein, the binding ligands include proteins or small molecules. For example, when the analyte is an enzyme, suitable binding ligands include substrates, inhibitors, and other proteins that bind the enzyme, i.e. components of a multi-enzyme (or protein) complex. As will be appreciated by those in the art, any two molecules that will associate, may be used, either as the analyte or the binding ligand. Suitable analyte/binding ligand pairs include, but are not limited to, antibodies/antigens, receptors/ligand, proteins/nucleic acids; nucleic acids/nucleic acids, enzymes/substrates and/or inhibitors, carbohydrates (including glycoproteins and glycolipids)/lectins, carbohydrates and other binding partners, proteins/proteins; and protein/small molecules. These may be wild-type or derivative sequences. In a preferred embodiment, the binding ligands are portions (particularly the extracellular portions) of cell surface receptors that are known to multimerize, such as the growth hormone receptor, glucose transporters (particularly GLUT4 receptor), transferrin receptor, epidermal growth factor receptor, low density lipoprotein receptor, high density lipoprotein receptor, leptin receptor, interleukin receptors including IL-1, IL-2, IL-3, IL-4, IL-5, IL-6, IL-7, IL-8, IL-9, IL-11, IL-12, IL-13, IL-15 and IL-17 receptors, VEGF receptor, PDGF receptor, EPO receptor, TPO receptor, ciliary neurotrophic factor receptor, prolactin receptor, and T-cell receptors.

In another aspect of the invention, the system comprises at least one pump 160. These pumps can be any type of pump device including electrode based pumps. Electromechanical pumps can be used in the systems of the invention, e.g. based upon capacitive, thermal, and piezoelectric actuation. Suitable on chip pumps include, but are not limited to, electroosmotic (EO) pumps and electrohydrodynamic (EHD) pumps; these electrode based pumps have sometimes been referred to in the art as “electrokinetic (EK) pumps”. All of these pumps rely on configurations of electrodes placed along a flow channel. As is described in the art, the configurations for each of these electrode based pumps are slightly different; for example, the effectiveness of an EHD pump depends on the spacing between the two electrodes, with the closer together they are, the smaller the voltage required to be applied to effect fluid flow. Alternatively, for EO pumps, the spacing between the electrodes should be larger, with up to one-half the length of the channel in which fluids are being moved, since the electrode are only involved in applying force, and not, as in EHD, in creating charges on which the force will act.

In one embodiment, an electroosmotic pump is used. Electroosmosis (EO) is based on the fact that the surface of many solids, including quartz, glass and others, become variously charged, negatively or positively, in the presence of ionic materials. The charged surfaces will attract oppositely charged counterions in aqueous solutions. Applying a voltage results in a migration of the counterions to the oppositely charged electrode, and moves the bulk of the fluid as well. The volume flow rate is proportional to the current, and the volume flow generated in the fluid is also proportional to the applied voltage. Electroosmostic flow is useful for liquids having some conductivity and generally not applicable for non-polar solvents.

In another embodiment, an electrohydrodynamic (EHD) pump is used. In EHD, electrodes in contact with the fluid transfer charge when a voltage is applied. This charge transfer occurs either by transfer or removal of an electron to or from the fluid, such that liquid flow occurs in the direction from the charging electrode to the oppositely charged electrode. EHD pumps can be used to pump resistive fluids such as non-polar solvents.

In another aspect, the pumps are external to the microfluidic device or chamber 100. In this aspect, the pump may be a peristaltic pump, syringe pump or other pump commonly used in the art.

In another aspect of the invention, the devices of the invention include at least one fluid valve 130 that can control the flow of fluid into or out of a module or chamber of the device or divert the flow into one or more channels. A variety of valves are known in the art. For example, in one embodiment, the valve may comprise a capillary barrier, as generally described in PCT US97/07880, incorporated by reference. In this embodiment, the channel opens into a larger space designed to favor the formation of an energy minimizing liquid surface such as a meniscus at the opening. Typically, capillary barriers include a dam that raises the vertical height of the channel immediately before the opening into a larger space such a chamber. In addition, as described in U.S. Pat. No. 5,858,195, incorporated herein by reference, a type of “virtual valves” can be used.

In yet another embodiment, the devices of the invention include sealing ports, to allow the introduction of fluids, including samples, into any of the modules of the invention, with subsequent closure of the port to avoid the loss of the sample.

The devices of the invention can include at least one storage modules for assay reagents (e.g., buffer, sample, binding agent). These are connected to other modules of the system using flow channels and may comprise wells or chambers, or extended flow channels. They may contain any number of reagents, buffers, enzymes, electronic mediators, salts, and the like, including freeze dried reagents.

In another embodiment, the devices of the invention include a mixing module; again, as for storage modules, these may be extended flow channels (particularly useful for mixing), wells or chambers. Particularly in the case of extended flow channels, there may be protrusions on the side of the channel to cause mixing.

The devices of the invention include a detection module. In one aspect, a separate detection module creates additional flexibility to employ signal enhancement, that is “signal amplification” protocols. For example, the detection module can incorporate both electrical sensing and optical illumination to enable a scheme where the label probes include multiple detection moieties that are photochemically dissociated to amplify the detected signal from a single probe above the background threshold.

Where the target analyte is a nucleic acid molecule, the detection module can be based upon techniques described in U.S. Pat. Nos. 5,591,578; 5,824,473; 5,770,369; 5,705,348 and 5,780,234; U.S. Ser. Nos. 09/096,593; 08/911,589; 09/135,183; and 60/105,875; and PCT applications US97/20014 and US98/12082; all of which are hereby incorporated by reference in their entirety. The system is generally described as follows. A target analyte is introduced to the interaction chamber where the target analyte interacts with a binding ligand present on a substrate to form a complex. In general, there are two basic detection mechanisms. In one embodiment, detection is based on electron transfer through the stacked π-orbitals of double stranded nucleic acid. This basic mechanism is described in U.S. Pat. Nos. 5,591,578, 5,770,369, and 5,705,348 and PCT US97/20014. Briefly, electron transfer can proceed rapidly through the stacked π-orbitals of double stranded nucleic acid, and significantly more slowly through single-stranded nucleic acid. Accordingly, this can serve as the basis of an assay.

This may be done where the target analyte is a nucleic acid; alternatively, a non-nucleic acid target analyte is used, with an optional capture binding ligand (to attach the target analyte to the detection electrode) and a soluble binding ligand that carries a nucleic acid “tail”, that can then bind either directly or indirectly to a detection probe on the surface to effect detection.

In one aspect, the detection modules of the invention comprise electrodes. By “electrode” herein is meant a composition, which, when connected to an electronic device, is able to sense a current or charge and convert it to a signal. Alternatively an electrode can be defined as a composition which can apply a potential to and/or pass electrons to or from species in the solution. Electrodes are known in the art and include, but are not limited to, certain metals and their oxides, including gold; platinum; palladium; silicon; aluminum; metal oxide electrodes including platinum oxide, titanium oxide, tin oxide, indium tin oxide, palladium oxide, silicon oxide, aluminum oxide, molybdenum oxide (Mo₂O₆), tungsten oxide (WO₃) and ruthenium oxides; and carbon (including glassy carbon electrodes, graphite and carbon paste).

In another aspect, the detector can be an optical detector capable of detecting an optical change. The change in optics may be the result of the presence of a luminescence or fluorescence label bound to the complex 90. For example, the binding ligand can interact with a target analyte within the interaction chamber 100 to generate a bound analyte 80. The bound analyte 80 is exposed to a labeled binding domain 55 comprising a binding domain 50 and a detectable label 60. In this aspect, the detectable label is a luminescent or fluorescent molecule.

In another aspect, can include any of a variety of known sensors, including, for example, surface acoustic wave sensors, quartz crystal resonators, metal oxide sensors, dye-coated fiber optic sensors, dye-impregnated bead arrays, micromachined cantilever arrays, composites having regions of conducting material and regions of insulating organic material, composites having regions of conducting material and regions of conducting or semiconducting organic material, chemically-sensitive resistor or capacitor films, metal-oxide-semiconductor field effect transistors, bulk organic conducting polymeric sensors, and other known sensor types. Techniques for fabricating particular sensor types are disclosed in Ballantine, D. S.; Rose, S. L.; Grate, J. W.; Wohltjen, H. Anal. Chem. 1986, 58, 3058; Grate, J. W.; Abraham, M. H. Sens. Actuators B 1991, 3, 85; Grate, J. W.; Rosepehrsson, S. L.; Venezky, D. L.; Klusty, M.; Wohltjen, H. Anal. Chem. 1993, 65, 1868; Nakamoto, T.; Fukuda, A.; Moriizumi, T. Sens. Actuators B 1993, 10, 85 (surface acoustic wave (SAW) devices), Gardner, J. W.; Shurmer, H. V.; Corcoran, P. Sens. Actuators B 1991, 4, 117; Gardner, J. W.; Shurmer, H. V.; Tan, T. T. Sens. Actuators B 1992, 6, 71; Corcoran, P.; Shurmer, H. V.; Gardner, J. W. Sens. Actuators B 1993, 15, 32 (tin oxide sensors), Shurmer, H. V.; Corcoran, P.; Gardner, J. W. Sens. Actuators B 1991, 4, 29; Pearce, T. C.; Gardner, J. W.; Friel, S.; Bartlett, P. N.; Blair, N. Analyst 1993, 118, 371 (conducting organic polymers), Freund, M. S.; Lewis, N. S. Proc. Natl. Acad. Sci 1995, 92, 2652 (materials having regions of conductors and regions of insulating organic material), White, J.; Kauer, J. S.; Dickinson, T. A.; Walt, D. R. Anal. Chem. 1996, 68, 2191 (dye-impregnated polymer films on fiber optic sensors), Butler, M. A.; Ricco, A. J.; Buss, R. J. Electrochem. Soc. 1990, 137, 1325; Hughes, R. C.; Ricco, A. J.; Butler, M. A.; Pfeifer, K. B. J. Biochem. and Biotechnol. 1993, 41, 77 (polymer-coated micromirrors), Slater, J. M.; Paynter, J. Analyst 1994, 119, 191; Slater, J. M.; Watt, E. J. Analyst 1991, 116, 1125 (quartz crystal microbalances (QCMs)), Keyvani, D.; Maclay, J.; Lee, S.; Stetter, J.; Cao, Z. Sens. Actuators B 1991, 5, 199 (electrochemical gas sensors), Zubkans, J.; Spetz, A. L.; Sundgren, H.; Winquist, F.; Kleperis, J.; Lusis, A.; Lundstrom, I. Thin Solid Films 1995, 268, 140 (chemically sensitive field-effect transistors) and Lonergan, M. C.; Severin, E. J.; Doleman, B. J.; Beaber, S. A.; Grubbs, R. H.; Lewis, N. S. Chem. Mater. 1996, 8, 2298 carbon black-polymer composite chemiresistors).

In one embodiment, electronic detection is used, including amperommetry, voltammetry, capacitance, and impedence. Suitable techniques include, but are not limited to, electrogravimetry; coulometry (including controlled potential coulometry and constant current coulometry); voltametry (cyclic voltametry, pulse voltametry (normal pulse voltametry, square wave voltametry, differential pulse voltametry, Osteryoung square wave voltametry, and coulostatic pulse techniques); stripping analysis (aniodic stripping analysis, cathiodic stripping analysis, square wave stripping voltammetry); conductance measurements (electrolytic conductance, direct analysis); time-dependent electrochemical analyses (chronoamperometry, chronopotentiometry, cyclic chronopotentiometry and amperometry, AC polography, chronogalvametry, and chronocoulometry); AC impedance measurement; capacitance measurement; AC voltametry; and photoelectrochemistry.

The devices of the invention are used to detect target analytes in samples. As described herein, target analytes interact (e.g., bind, hybridize, and the like) with binding ligands. As will be appreciated by those in the art, a large number of analytes may be detected using the methods, devices and systems of the invention; basically, any target analyte for which a binding ligand may be made may be detected using the invention.

In one aspect, the target analyte is a nucleic acid (e.g., a polynucleotide or oligonucleotide). Typically a nucleic acid in a biological sample will comprise phosphodiester bonds, however, nucleic acids may comprise a modified backbone comprising, for example, phosphoramide, phosphorothioate, phosphorodithioate, O-methylphophoroamidite linkages, and peptide nucleic acid backbones and linkages. Other analog nucleic acids include those with positive backbones; non-ionic backbones and non-ribose backbones. Nucleic acids containing one or more carbocyclic sugars are also included within the definition of nucleic acids. These modifications of the ribose-phosphate backbone may be done to facilitate the addition of electron transfer moieties, or to increase the stability and half-life of such molecules in solution. As will be appreciated by those in the art, all of these nucleic acid analogs may find use in the invention, particularly as binding ligands. In addition, mixtures of naturally occurring nucleic acids and analogs can be made.

For example, in one aspect of the invention a binding ligand on a solid surface of the invention comprises a nucleic acid. The nucleic acid binding ligands may be in the form of an array on the solid support. In some embodiments, the arrayed nucleic acids comprise an array of complementary target nucleic acid capable of hybridizing to a target nucleic acid. In yet other embodiments, the nucleic acid comprises a sequence that interacts with a DNA binding protein.

In some embodiments, the array of nucleic acids comprises at least 20, at least 50, at least 100, or at least 1000 or more distinct nucleic acid molecules, the nucleic acids may be the same or different. Where the solid support comprises nucleic acids, the target analyte can be a polypeptide or nucleic acid molecule.

Peptide nucleic acids (PNA) which includes peptide nucleic acid analogs can be used in the methods and compositions of the invention. Such peptide nucleic acids have increased stability and are useful as binding ligands. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched basepairs. DNA and RNA typically exhibit a 2-4° C. drop in Tₘ for an internal mismatch. With the non-ionic PNA backbone, the drop is closer to 7-9° C. This allows for better detection of mismatches. Similarly, due to their non-ionic nature, hybridization of the bases attached to these backbones is relatively insensitive to salt concentration.

The nucleic acids may be single stranded or double stranded, or contain portions of both double stranded or single stranded sequence. The nucleic acid may be DNA, both genomic and cDNA, RNA or a hybrid, where the nucleic acid contains any combination of deoxyribo-and ribo-nucleotides, and any combination of bases, including uracil, adenine, thymine, cytosine, guanine, inosine, xathanine hypoxathanine, isocytosine, isoguanine, and the like. Such nucleic acids comprise nucleotides and nucleoside and nucleotide analogs, and modified nucleosides such as amino modified nucleosides. In addition, “nucleoside” includes non-naturally occurring analog.

The invention provides methods of detecting target nucleic acids in a sample. Where the solid support comprises nucleic acids, the target analyte will typically be a nucleic acid. The target nucleic acid may be a portion of a gene, a regulatory domain, genomic DNA, cDNA, RNA including mRNA and rRNA, and the like. The target nucleic acid may be any length, with the understanding that longer molecules are more specific. In some embodiments, it may be desirable to fragment or cleave nucleic acids in a sample. As is outlined more fully below, binding ligands comprising nucleic acids are made to hybridize to target nucleic acids to determine the presence or absence of the target nucleic acid in a sample.

In another embodiment, the solid support comprises polypeptides linked to the solid support. The polypeptide can be an antibody, a receptor ligand, a receptor, a polypeptide epitope, and the like. A solid support can comprise an array of polypeptides. In some embodiments, the array comprises at least 20, at least 50, at least 100, or at least 1000 or more, distinct polypeptides, the polypeptides may be the same of different. Where the solid support comprises polypeptides, the target analyte includes, but is not limited to, a peptide, a polypeptide, a nucleic acid, a carbohydrate, a lipid and a small molecule.

The target analyte (e.g., the target nucleic acid or polypeptide) comprises different target domains. For example, in “complex” assays as described herein, a binding ligand on the solid surface interacts with a first target domain of the sample target analyte. Where the binding ligand is a nucleic acid, the binding ligand hybridizes to a first target domain on a target nucleic acid. A labeled binding ligand then hybridizes to a second target domain on the target nucleic acid. Similarly, where the binding ligand is a polypeptide (e.g., an antibody), a binding ligand on the solid surface interacts with a first epitope on a target analyte and a labeled binding ligand interacts with a second epitope on the target analyte. The target domains may be adjacent (i.e. contiguous) or separated.

Label 60 can be any detectable label including, but are not limited to, any composition detectable by electrical, optical, spectrophotometric, photochemical, biochemical, immunochemical, and/or chemical techniques. For example, label 60 includes conducting, luminescent, fluorescent, chemiluminescent, bioluminescent and phosphorescent labels, nanoparticles, metal nanoparticles, gold nanoparticles, silver nanoparticles, chromogens, antibodies, antibody fragments, enzymes, substrates, cofactors, inhibitors, binding proteins, magnetic particles and spin labels.

Examples of photodetectable labels that can be used in the methods and system of the invention include dansyl chloride, rhodamine isothiocyanate, TRIT (tetramethyl rhodamine isothiol), NBD (7-nitrobenz-2-oxa-1,3-diazole), Texas Red, phthalic acid, terephthalic acid, isophthalic acid, cresyl fast violet, cresyl blue violet, brilliant cresyl blue, para-aminobenzoic acid, erythrosine, biotin, digoxigenin, fluorescein, 5-carboxy-4′,5′-dichloro-2′,7′-dimethoxy fluorescein, 5-carboxy-2′,4′,5′,7′-tetrachlorofluorescein, 5-carboxyfluorescein, 5-carboxyrhodamine, aminoacridine, 6-carboxyrhodamine, 6-carboxytetramethyl amino phthalocyanines, azomethines, cyanines, xanthines, succinylfluoresceins, rare earth metal cryptates, europium trisbipyridine diamine, a europium cryptate or chelate, diamine, dicyanins, La Jolla blue dye, allopycocyanin, allococyanin B, phycocyanin C, phycocyanin R, thiamine, phycoerythrocyanin, phycoerythrin R, luciferin, or acridinium esters. Such labels can be obtained from commercial sources such as Molecular Probes and attached to amino acids and nucleic acids by methods known in the art.

In other embodiments of the invention, electrically detectable labels can be used including, e.g., metal nanoparticles. For example, gold or silver nanoparticles of between 1 nm and 3 nm in size may be used, although nanoparticles of different dimensions and mass may also be used. Methods of preparing nanoparticles are known in the art or are commercially available. Nanoparticles may be attached covalently to amino acid residues either before or after the amino acid residues are incorporated into a binding ligand or other polypeptide.

Methods used for immobilizing polypeptide or nucleic acids to a solid support are described in the following references, and others (Mosbach (1976) Meth. Enzymol. 44:2015-2030; Hermanson, G. T. (1996) Bioconjugate Techniques, Academic Press, NY; Bickerstaff, G. (ed) (1997) Immobilization of Enzymes and Cells, Humana Press, NJ; Cass and Ligler (eds.) (1998) Immobilized Biomolecules in Analysis, Oxford University Press; Watson et al. (1998) Curr. Opin. Biotech. 609:614; Ekins (1998) Clin. Chem. 44:2105-2030; Roda et al. (2000) Biotechniques 28:492-496; Wong (1993) Chemistry of Protein Conjugation and Cross-linking CRC Boca Raton, Fla.; Taylor, (1991) Protein Immobilization:fundamentals and applications Marcel Dekker, Inc New York; Hutchens (ed) (1989) Protein recognition of immobilized ligands, Vol 83 Alan R Liss, Inc; Sleytr U. B. (ed) (1993) Immobilized macromolecules, application potentials Vol 51. Springer series in applied biology, SpringerVerlag, London; Wilchek and Bayer (eds) (1990) Avidin-Biotin Technology. Academic Press, San Diego; Ghosh et al. (1987) Nucleic Acids Res. 15:5353-5372; Burgener et al. (2000) Bioconjug. Chem. 11:749-754; Steel et al. (2000) Biophys J 79:975-981; Afanassiev et al. (2000) Nucleic Acids Res. 28:E66; Roda et al. (2000) Biotechniques 28:492-496; Shena (ed.) (2000) DNA Microarrays, a practical approach (Oxford University Press); Schena (ed.) (2000) Microarray Biochip Technology. (Eaton Publishing Natick, Mass.); MacBeath et al. (2000) Science 289:1760-1763; Schena et al. (1998) Trends in Biotechnol. 16:301-306; and Ramsey (1998) Nat. Biotechnol. 16:40-44; all of which are incorporated by reference herein.

Many coupling agents are known in the art and can be used to immobilize biomolecules in the current invention. Over 300 cross-linkers are currently available. These reagents are commercially available (e.g., from Pierce Chemical Company (Rockford, Ill.)). A cross-linker is a molecule which has two reactive groups with which to covalently attach a protein, nucleic acids or other molecules. In between the reactive groups is typically a spacer group. Steric interference with the activity of the biomolecule by the surface may be ameliorated by altering the spacer composition or length. In addition, the linker can comprise cleavage sites for enzymes to dissociate the binding ligand from the solid support. There are two groups of cross-linkers, homobifunctional and heterobifunctional. In the case of heterobifunctional crosslinkers, the reactive groups have dissimilar functionalities of different specificities. On the other hand, homobifunctional cross linkers' reactive groups are the same. A thorough review of cross-linking can be found in Wong, 1993, Chemistry of Protein Conjugation and Cross-linking, CRC Press, Boca Raton.

When macromolecular ligands are used, the biomolecules should be immobilized in such a way as to reduce steric hindrances generated by the support. A variety of methods for achieving this are known in the art. For example, the active site or other binding region of the biomolecule can be orientated away from the surface (Reviewed in Bickerstaff, (ed.) (1997) Immobilization of Enzymes and Cells, pp. 261-275). Suitable spacer arms may include, but are not limited to, carbon spacers, poly ethylene glycol polymers, peptides, dextrans, proteins, and nucleic acids. For example, Maskos et al. (1992) teach methods of immobilizing oligonucleotides to chips.

Other methods of protein immobilization suitable for immobilizing polypeptides involves immobilization via a fusion tail. Fusion proteins are commonly constructed having fusion tail systems to promote efficient recovery, purification, and immobilization of recombinant proteins (reviewed in Ford, et al. (1991) Protein Expr. Purif. 2:95-107). A binding ligand can be genetically engineered to contain a C- or N-terminal polypeptide tail, which may act as a spacer arm and provides the biochemical basis for specificity in purification and/or immobilization. Tails with a variety of characteristics have been used. Examples include entire proteins or protein domains with affinity for immobilized ligands, a biotin-binding domain for in vivo biotination promoting affinity of the fusion protein to avidin or streptavidin, peptide binding proteins with affinity to immunoglobulin G or albumin, carbohydrate-binding proteins or domains, antigenic epitopes with affinities for monoclonal antibodies, charged amino acids for use in charge-based recovery methods, poly(His) residues for recovery by immobilized metal affinity chromatography. For example, Ribeiro et al. (1995) Anal. Biochem. 228, 330-335, genetically engineered elongation factor Tu from Thermus thermophilus creating a protein having a spacer of nine amino acids followed by six histidine residues on its C-terminus. This protein is immobilized on Ni²⁺-nitriloacetic acid agarose.

The binding ligands may be immobilized to the solid support by covalent or noncovalent attachment. These molecules may be immobilized, for example, using chemical cross-linkers to covalently attach them to a surface, by adsorption, entrapment, encapsulation, or by binding to a protein, nucleic acid, or peptide nucleic acid. For example, a binding ligand may be immobilized by electrostatic binding to molecules such as poly-L-lysine. Alternatively, the binding ligand may optionally be cross-linked to a suitable spacer arm (e.g., one that facilitates desorption from the support) and attached to a solid support. Biotinylated binding agents may be immobilized by binding to avidin or streptavidin on the solid support.

The process of programmed desorption (e.g., for temporal synchronous detection) can be done by photochemically-induced release as well as by electrochemical release. For the former, an appropriate photoactive linker would be incorporated either into the capture probe tether to the solid support, or the label probe tether to the affinity binding site on the capture probe (either an antibody, aptamer, complementary oligomer, and the like) In the latter case only the label would undergo programmed release. (If a positive signal is detected from the released labels, the analyte could be released and captured in a subsequent step—if desired.)

In one aspect, binding ligands can be adsorbed, embedded or entrapped or covalently linked to surfaces. The binding ligand (e.g., a polypeptide) can be adsorbed or attached to nanoparticles, for example, and these nanoparticles can be position in microflow channels. The nanoparticles can be held in position using magnetic nanoparticles and magnetic force or by a filter, grid or other support. Alternatively, the proteins can be adsorbed or covalently attached to the surfaces within the microflow channels or wells.

The binding ligand can be immobilized on the surface within a microflow channel, well or membrane, or can be immobilized onto the surfaces of beads, membranes or transducers or other surfaces placed in a flow channel, chamber or well. Suitable beads for immobilization of polypeptides or nucleic acids include chemically or physically crosslinked gels and porous or nonporous resins such as polymeric or silica based resins. Suitable media for adsorption include, without limitation, ion exchange resins, hydrophobic interaction compounds, sulfhydryls and inherently active surfaces and molecules such as plastics or activated plastics, aromatic dye compounds, antibodies, antibody fragments, aptamers, oligonucleotides, metals or peptides. Examples of some suitable commercially available polymeric supports include, but are not limited to, polyvinyl, polyacrylic and polymethacrylate resins.

Many coupling agents are known in the art and can be used to immobilize biomolecules in the methods and devices of the invention. Coupling agents are exemplified by bifunctional crosslinking reagents, i.e., those which contain two reactive groups which may be separated or tethered by a spacer. These reactive ends can be of any of a number of functionalities including, without limitation, amino reactive ends such as N-hydroxysuccinamide, active esters, imidoesters, aldehydes, epoxides, sulfonyl halides, isocyanate, isothiocyanate, nitroaryl halides, and thiol reactive ends such as pyridyl disulfide, maleimides, thiophthalimides and active halogens.

Printing methods for making arrays can be used to deliver nucleic acid or polypeptides to surfaces in predetermined locations. For example, aminophenyl-trimethoxysilane treated glass surfaces can bind 5′ amino-modified oligonucleotides nucleic acids using a homobifunctional crosslinker to attach the aminated oligonucleotide to the aminated glass as taught in Guo et al. (1994) Nucleic Acids Research 22:5456-5465. Another known method for arraying nucleic acids is to react the nucleic acid with succinic anhydride and attach the resulting carboxylate group via an ethyldimethylaminopropylcarbodiimide-mediated coupling reaction (Joos et al. (1997) Anal. Biochem. 247:96-101). In another method 5′ phosphate modified nucleic acids react with imidazole to produce a 5′-phosphoimidazolide that can bind to surface amino groups via a phosphoramidate linkage (Chu et al. (1983) Nucleic Acids Research 11:6513-6529). The linker is typically long enough to eliminate steric hindrances caused by the solid support surface. For example, Shchepinov et al. (1997) Nucleic Acids Research 25:1155-1161, reported that an optimal spacer length of about 40 atoms long will increase binding yields by 150-fold in nucleic acid hybridization assays.

A variety of hybridization conditions may be used in the invention to facilitate binding of a target analyte to a binding agent on a substrate, including high, moderate and low stringency conditions; see for example Maniatis et al., Molecular Cloning: A Laboratory Manual, 2d Edition, 1989, and Short Protocols in Molecular Biology, ed. Ausubel, et al., hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH and nucleic acid concentration) at which 50% of the probes complementary to the target sequence at equilibrium (as the target sequences are present in excess, at Tm, 50% of the probes are occupied at equilibrium). Stringent conditions will be those in which the salt concentration is less than about 1.0 sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g., greater than 50 nucleotides). Stringent conditions may also be achieved with the addition of destabilizing agents such as formamide. The hybridization conditions may also vary when a non-ionic backbone, i.e. PNA is used, as is known in the art. In addition, cross-linking agents may be added after target binding to cross-link, i.e. covalently attach, the two strands of the hybridization complex.

Thus, the assays are generally run under stringency conditions which allows formation of the hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, and the like.

Accordingly, the invention provides a device for the detection of target analytes comprising a solid substrate. The solid substrate can be made of a wide variety of materials and can be configured in a variety of designs. In addition, a device may comprise one or more solid supports (see, e.g., FIG. 3). In some cases, a portion of the substrate may be removable; for example, the solid support/interaction chamber may be a detachable cassette that can be removed from the device following use.

The devices of the invention can be made in a variety of ways, as will be appreciated by those in the art. Suitable fabrication techniques again will depend on the choice of substrate. Exemplary methods include, but are not limited to, a variety of micromachining and microfabrication techniques, including film deposition processes such as spin coating, chemical vapor deposition, laser fabrication, photolithographic and other etching techniques using either wet chemical processes or plasma processes, embossing, injection molding and bonding techniques. In addition, there are printing techniques for the creation of desired fluid guiding pathways; that is patterns of printed material can permit directional fluid transport.

In one embodiment, the solid substrate is configured for handling a single sample that may contain a plurality of target analytes. That is, a single sample is added to the device and the sample may either be aliquoted for parallel processing for detection of the analytes or the sample may be processed serially, with individual targets being detected in a serial fashion. In addition, samples may be removed periodically or from different locations for in line sampling.

In addition, it should be understood that while most of the discussion herein is directed to the use of planar substrates with microchannels and wells, other geometries can be used as well. For example, two or more planar substrates can be stacked to produce a three dimensional device, that can contain microchannels flowing within one plane or between planes; similarly, wells may span two or more substrates to allow for larger sample volumes. Thus, for example, both sides of a substrate can be used to attach molecules of interest.

The working examples below are provided to illustrate, not limit, the invention. Various parameters of the scientific methods employed in these examples are described in detail below and provide guidance for practicing the invention in general.

### EXAMPLE

FIG. 3 shows one specific representation exemplifying how EAID biosensors may be configured. This specific example is one possible representation of EAID biosensors that could measure the ratio between free and complexed PSA in solution. However, the sensor architecture is completely general—as many parallel capture arrays as necessary can be incorporated, each, for example, specifically functionalized to capture a particular analyte in the solution to be analyzed.

From left to right the components are as follows: B=reservoir for buffer solution, A=reservoir for the solution to be analyzed (presumably containing the analyte), L=reservoir for label/capture complex, circles encompassing (x)=electrically-actuated fluidic valves, heavy lines=fluidic channels, light lines=electrical connections, P1 and P2=electrically-actuated fluidic pumps, exh=fluidic exhaust ports.

By supplying the smallest possible amount of buffer to drive this process, target analyte concentration can be maintained as high as possible.

In Step 1 buffer is electrically-pumped from the buffer reservoir and forces the analyte solution to the capture arrays which have been previously specifically-biofunctionalized so as to immobilize any target analyte in the solution presented for analysis. If necessary, the solution can be passed through a filtration element to, for example, remove high levels of background immunoglobulins and albumin.

Another optional process step in the analysis process is recirculation of the analyte solution. This is accomplished by valving off fluidic components that are extraneous to this step, and employing a second electrically-actuated pump to drive the solution around a fluidic loop that incorporates the capture arrays. (It can optionally also incorporate a filtration element if necessary.) This permits repeated encounters between the capture chemistry and the target analytes to enhance the probability of capture.

The captured analytes is then labeled with an entity that will, subsequently, greatly enhance their detection probability. This process begins by electrically-pumping buffer through the reservoir containing the label complexes (L). This forces the labels into the chambers containing the capture arrays. (If deemed acceptable the label complexes for all of the specific target analytes can be introduced in a single step. If necessary, however, the label complexes for the different analytes could be introduced sequentially by incorporating more valves, lines, and label reservoirs into the fluidic “logic”.) Use of the smallest possible amount of buffer in this step maximizes the concentration of the label complexes in solution, and thereby enhances the probability of their binding to the analyte. Optionally, the fourth step in the analysis process is recirculation of the solution containing the label complexes. This permits further enhancement of the binding probability of the label complex to the analyte

The process may optionally include a flushing of the capture arrays to eliminate all species that are not specifically bound by the capture probes. If necessary this flush could be promoted by a specific chemical agent. The flushing process, as for the other steps, can be effected under electrical control by actuation of the electrically-driven pump.

A calibrated, steady flow rate of buffer solution leading from the capture arrays to the detector is performed. This permits a known transit time for the analytes from the capture pads to the detector. This transit time could be directly measured by release of a test label from a pad specifically included in the array to provide a test “calibration” signal. For example, calibration can be performed using a known amount of bound label probe—which can released upon command to precalibrate the time for detection.

As described above, the capture array be selectively addressed to make the release of certain bound analytes readily detectable. For example, referring to FIG. 3 there is shown the selective release of a target at position CA-1(a) can be performed and the travel time an detection of a only a first addressed portion of the binding array can be made. A timed release and subsequent detection of the target analytes can be accomplished. A pulsatory stimulus—for example, electrical or optical—is applied to a given capture pad. The released label/analyte/ligand complex flows to the detector in a precalibrated time, and is observed using a detection process synchronized to the release stimulus, delayed by a pre-calibrated amount. This delay may simply be based upon the average flow velocity of particles in solution and the pre-measured array-detector path length, l. Alternatively, this may be directly measured by a test release from a calibration pad incorporated within each capture array. The synchronous detection process is illustrated in the top left inset.

For example, the formation of mixed self-assembled monolayers (SAMs) containing biotinylated alkylthiols (BAT) and triethyleneglycol alkylthiols (TEG) in aqueous solvents on Au can be performed to reversibly link a binding moiety to a solid support. Solid supports comprising, for example, Au, can be recycled using electrochemical protocols for repeated SAM formation. The use of aqueous solvents in these techniques and studies is useful for the development of microfluidic-based biosensors. In addition, aqueous solvents are closer and more compatible with physiological conditions for biological (cells and protein) samples than organic solvents. The capability to transition from organic solvents to aqueous solvents for the formation of SAMs inside a biosensor device reduces the risk of buffer salt precipitation, polymer swelling, and toxicity to biological samples. Comparison of fluorescence data between solution mixtures of 0.1 mM alkylthiols in varied concentrations of ethanol and water solvent indicates that functional SAMs may be adsorbed from ethanolic solvent compositions as low as 1% in water. In addition, cyclic voltammetry (CV) data indicate that SAMs adsorbed in aqueous solution form well-ordered monolayers with few pinholes faster than SAM formation in ethanol. Protein binding assays performed with Cy3-labled streptavidin demonstrate that cleanliness of the gold substrate assists in the formation of quality monolayers. Samples cleaned with oxidative potentials under CV conditions formed better SAMS that exhibited higher fluorescence signals in the protein-binding assay. In addition, the application of oxidative potentials on SAM-coated Au results in the oxidation and removal of thiolates from the surface. Using these methods, well formed SAMs may be formed in aqueous solution at the reduced incubation time of 60 minutes and removed in as few as 15 seconds by applying oxidative potentials. Thus, such techniques are useful for the formation and removal of alkylthiolate monolayers on substrates in the context of a microfluidics based biosensor. SAMs formed using the aqueous solutions explored here generate biosensor substrates faster and in an environment that is amenable to biological samples, namely aqueous environments.

A number of embodiments have been described. Nevertheless, it will be understood that various modifications may be made without departing from the spirit and scope of the description. Accordingly, other embodiments are within the scope of the following claims.

What is claimed is:

1. A system for the detection of a target analyte in a fluid sample comprising:
a capture chamber comprising:
an inlet port;
an outlet port; and
at least one solid support member, wherein the solid support member is disposed between and in fluid communication with the inlet and outlet port, the at least one solid support member comprising at least one binding ligand specific for the target analyte;
a sample reservoir, fluidly connected to the inlet port;
a labeled binding ligand reservoir, fluidly connected to the inlet port;
a detector, fluidly connected to the outlet port; and
means for controlling the timed release and flow of a binding ligand from the at least one solid support to the detector.

2. The system of claim 1, further comprising a buffer reservoir fluidly connected to the capture chamber.

3. The system of claim 1, further comprising a label mixing chamber in fluid communication with the inlet port of the capture chamber.

4. The system of claim 3, further comprising a buffer reservoir fluidly connected to the label mixing chamber and the inlet port of the capture chamber.

5. The system of claim 1, further comprising a filter adapted for the removal of contaminants and in fluid communication with the sample reservoir.

6. The system of claim 1, wherein the at least one solid support comprises a plurality of binding ligands.

7. The system of claim 6, wherein the plurality of binding ligands comprise an array of binding ligands.

8. The system of claim 1, comprising a means for inducing fluid flow through the system.

9. The system of claim 8, wherein said means for inducing flow comprises at least one pump.

10. The system of claim 1, further comprising a plurality of valves operational to cause, stop or reduce fluid flow through the system.

11. The system of claim 1, wherein the at least one binding ligand is removably attached to the at least one solid support.

12. The system of claim 11, wherein said binding ligand is a nucleic acid.

13. The system of claim 11, wherein the binding ligand comprises a polypeptide.

14. The system of claim 13, wherein the polypeptide comprises an antibody or fragment thereof.

15. The system of claim 11, wherein the binding ligand is attached by a techniques selected from the group consisting of photochemical, chemical, photoelectrical, and dielectrophoretic means.

16. The system of claim 15, wherein the binding ligand is removably attached to the at least one solid support by electrophoretic forces, electrochemical means, photochemical means, or enzymatic means.

17. A method for the detection of a target analyte in a fluid sample comprising:
contacting the sample with at least one solid support member comprising at least one first binding ligand removably attached to the solid support, wherein the at least one first binding ligand is specific for the target analyte, wherein the target analyte interacts with the binding ligand to form a bound analyte;
contacting the bound analyte with a second binding ligand comprising a label, wherein the second binding ligand interacts with the target analyte to form a complex;
dissociating the complex from the solid support; and
detecting the label on the complex.

18. The method of claim 17, wherein the at least one solid support comprises a plurality of binding ligands.

19. The method of claim 18, wherein the plurality of binding ligands comprise an array of binding ligands.

20. The method of claim 17, wherein the method is performed in a microfluidic device.

21. The method of claim 20, wherein the detecting is performed at a time predicted based upon a flow rate from the at least one solid support to a detector and the distance from the solid substrate to a detector.

22. The method of claim 17, wherein said binding ligand is a nucleic acid.

23. The method of claim 17, wherein the binding ligand comprises a polypeptide.

24. The method of claim 23, wherein the polypeptide comprises an antibody or fragment thereof.

25. The method of claim 17, wherein the binding ligand is removably attached by a technique selected from the group consisting of photochemical, chemical, photoelectrical, and dielectrophoretic.

26. The method of claim 17, wherein the binding ligand is removably attached to the at least one solid support by electrophoretic forces, electrochemical means, photochemical means, or enzymatic means.
